(19) United States
(12) Patent Application Publication
(10) Pub. No.: US 2023/0372076 A1
(43) Pub. Date: Nov. 23, 2023

(54) IMPLANT FOR PELVIC AND HIP PLASTY

(71) Applicants: Yougun WON, Seoul (KR); Tae Jin LEE, Seoul (KR); BIOCOEN CO., LTD., Yongin-si, Gyeonggi-do (KR)

(72) Inventors: Won Don YANG, Seoul (KR); Yougun WON, Seoul (KR); Tae Jin LEE, Seoul (KR)

(21) Appl. No.: 18/030,337

(22) PCT Filed: Oct. 5, 2021
(86) PCT No.: PCT/KR2021/013606

§ 371 Date: Apr. 5, 2023

(30) Foreign Application Priority Data
Oct. 5, 2020 (KR) 10-2020-0128279
Oct. 5, 2020 (KR) 10-2021-0045256

(51) Int. Cl. A61F 2/00

(52) U.S. Cl. CPC A61F 2/0059

(57) ABSTRACT

The present disclosure relates to an implant for pelvic and hip plasty, more particularly, to an implant for pelvic and hip plasty to increase a pelvis and hip volume of a human, the implant for pelvic and hip plasty includes an outer cover which has an interior space, includes an elastic material, and is to be placed in pelvis and hip tissues, a polymer gel filled in the interior space of the outer cover, and a flange portion which protrudes from an edge of the outer cover and is in close contact with and fixed to an iliac crest of a human.

### TECHNICAL FIELD

The present disclosure relates to an implant for pelvic and hip plasty.

### BACKGROUND ART

Generally, implants for plasty have been mainly used to correct shapes of or enlarge a nose, breasts, hips, etc. of female patients.

In this regard, a method of plastic surgery applied to a shape of hips or a saggy body part includes implantation of fat tissues, insertion of silicon gel implant, and insertion of silicon mold implant.

The implantation of fat tissues refers to a method of isolating and implanting fat tissues and is considered as the most ideal method. However, the implantation of fat tissues cannot be operated on thin patients. In addition, even when there is a sufficient amount of fat for collection is present in the body of a patient, there may still be a limitations depending on a degree of operation. Moreover, when an engraftment rate of a patient is low, it may be difficult to apply the implantation of fat tissues. That is, as there are various variables per each patient, the implantation has not been widely used in general cases due to surgical limitations.

A method using a silicon gel implant, etc. refer to a method of inserting in a surgical site an implant made by injecting a mixture of liquid silicon oil and/or physiological salt solution, etc. in a silicon bag, and characteristic softness of a product may lessens the foreign body sensation.

A silicon gel implant 1 may be inserted in a surgical site in hips 10 as illustrated in FIG. 1. In this case, the implant 1 may be inserted into a muscle 12 located between a bone 11 and fat 13 by surgery. Or, the implant 100 may be inserted in the fat 13 located between the muscle 12 and skin 14 by surgery. The implant 100 may be inserted in a proper position according to condition of body tissues of a patient.

When the implant 1 remains inserted in the body of a patent for a long time, the implant 1 may sag due to gravity, and accordingly, the original shape of the hips may not stay unchanged.

### DISCLOSURE

### Technical Problem

The present disclosure is devised to overcome the aforementioned issue, and more specifically, has a technical goal of providing an implant for pelvic and hip plasty which stays unsagged when inserted in body tissues for a long time.

### Technical Solution

The present disclosure is devised to overcome the aforementioned technical problem, and an implant for pelvis and hip plasty for increasing a pelvis and hip volume of a human includes:
a first implant body including an outer cover which has an interior space, includes an elastic material, and is to be placed in hip tissues under an iliac crest of the human,
a polymer gel filled in the interior space of the outer cover, and
a flange portion projecting from an edge of the outer cover and including an elastic-deformable material to be in close contact with and fixed to the iliac crest of the human; and
a fixing screw penetrating the flange portion and inserted into the iliac crest to fix the flange portion to the iliac crest,
wherein, when the flange portion is in contact with the iliac crest, the flange portion may deform into a shape corresponding to an outer shape of the iliac crest and is in close contact with the iliac crest.

In the implant for pelvic and hip plasty,
at least one first fastening hole may be formed in the flange portion such that the fixing screw fixed to the iliac crest of the human passes through the at least one first fastening hole.

In the implant for pelvic and hip plasty,
the flange portion may be bended to have a shape corresponding to a shape of the iliac crest.

In the implant for pelvic and hip plasty,
the flange portion may extend from an entire edge of the outer cover and form a ring shape.

In the implant for pelvic and hip plasty,
at least one flange portion may extend from a part of the edge of the outer cover.

In the implant for pelvic and hip plasty,
the flange portion may include the same material as the outer cover and may be integrated with the outer cover.

In the implant for pelvic and hip plasty,

the flange portion may have a shape corresponding to the iliac crest and may be fixed to the iliac crest.

In the implant for pelvic and hip plasty,
the polymer gel may include a silicon gel.

In the implant for pelvic and hip plasty,
the outer cover may include at least one of silicon elastomer and polyurethane.

In the implant for pelvic and hip plasty,
the outer cover may consist of an upper surface and a lower surface with respect to the flange portion, and the upper surface and the lower surface may have a convex shape.

The implant for pelvic and hip plasty may further include
a second implant body fixed to the iliac crest to extend a lateral width of a pelvis of the human and including a close contact surface having a shape corresponding to the iliac crest, and
the flange portion of the first implant body may be inserted and fixed between the close contact surface of the second implant body and a surface of the iliac crest.

In the implant for pelvic and hip plasty,
the second implant body may include
a base portion fixed onto the iliac crest of the pelvis and increasing a volume of the iliac crest, and
an extension extending from an edge of the base portion.

In the implant for pelvic and hip plasty,
a first fastening hole may be formed in the flange portion, and
a second fastening hole may be formed in the base portion of the second implant body at a position corresponding to the first fastening hole such that the fixing screw penetrates the first fastening hole and the second fastening hole and is fixed to the iliac crest to fix the first implant body and the second implant body to the iliac crest.

In the implant for pelvic and hip plasty,
a third fastening hole through which the fixing screw passes may be formed in the extension of the second implant body.

In the implant for pelvic and hip plasty,
the second implant body may include at least one material selected from a titanium alloy, a stainless alloy, polyethylene, and bio-ceramics.

According to the present disclosure devised to overcome the aforementioned technical problem, an implant for pelvic and hip plasty for increasing pelvis and hip volumes of a human, the implant for pelvic and hip plasty includes:
a first implant body including an outer cover which has an interior space, includes an elastic material, and is to be placed in hip body tissues,
a polymer gel filled in the interior space of the outer cover, and
a flange portion which projects from an edge of the outer cover and is in close contact with and fixed to the iliac crest of the human; and
a second implant body fixed to the iliac crest to extend a lateral width of a pelvis of the human and including a close contact surface having a shape corresponding to the iliac crest,
wherein the flange portion of the first implant body may be inserted and fixed between the close contact surface of the second implant body and a surface of the iliac crest.

### Advantageous Effects

In an embodiment of the present disclosure, provided is an implant for plasty including a first implant body having a flange portion protruding from an edge of an outer cover. The flange portion may be fixed to an iliac crest. In the implant for plasty according to the embodiment, as the flange portion protruding from the edge of the outer cover is fixed to the iliac crest, the implant may not sag by gravity over time after the surgery.

In addition, according to an embodiment of the present disclosure, the implant may increase a volume of the iliac crest as wells as a volume of hips.

### DESCRIPTION OF DRAWINGS

FIG. 1 is a diagram illustrating an existing implant for plasty placed in hips.

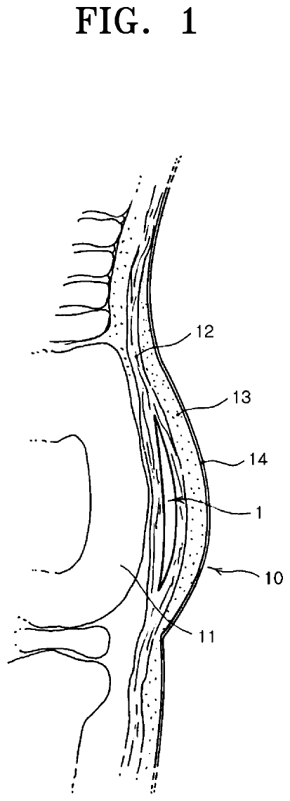

FIG. 2 is a perspective view illustrating a first embodiment of a first implant body of an implant for pelvic and hip plasty of the present disclosure.

FIG. 3 is a cross-sectional view taken along line III-III in FIG. 2.

FIG. 4 is a diagram illustrating placement of the first implant body of FIG. 2 onto a pelvis.

FIG. 5 is a diagram illustrating the first implant body of FIG. 2 placed onto a pelvis.

FIG. 6 is a diagram illustrating a second embodiment of a first implant body.

FIG. 7 is a cross-sectional view taken along line VII-VII in FIG. 6.

FIG. 8 is a diagram illustrating a third embodiment of an implant body.

FIG. 9 is a perspective view illustrating a second embodiment of an implant for pelvic and hip plasty of the present disclosure.

FIG. 10 is a diagram illustrating placement of a first implant body and a second implant body onto a pelvis.

FIG. 11 is a diagram illustrating a first implant body and a second implant body placed onto a pelvis.

### BEST MODE

### Mode for Invention

The embodiments of the present disclosure are provided to describe technical ideas of the present disclosure. The scope of rights according to the present disclosure is not limited to the embodiments provided below or detailed description thereof.

All technical terms used in the present disclosure have a meaning generally understood by a person skilled in the art, unless otherwise defined. All terms used in the present disclosure are selected for the purpose of describing the present disclosure more clearly, and are not selected to limit the scope of rights according to the present disclosure.

The expressions “comprise,” “include,” “have,” etc. used throughout the present disclosure should be understood as open-ended terms connoting the possibility of including other embodiments, unless otherwise mentioned in a phrase or a sentence in which the expression is used.

In the present disclosure, an expression used in the singular encompasses the expression of the plural, unless otherwise mentioned, and the same applies to the singular expressions used in the claims.

Such terms as “first,” “second,” etc., are used only to distinguish one component from another, and are not intended to limit an order or importance of the components.

Through of the present disclosure, when one component is “coupled to” or “connected to” another component, it should be construed as meaning that one component is directly connected to another component or one component is coupled or connected indirectly to another component via an intervening component arranged therebetween.

The expression “having a corresponding shape” used in the present disclosure means that a part of a flange portion in contact with an iliac crest has a identical or similar shape with the iliac crest, which leads to stable placement of an implant when the flange portion and the iliac crest are coupled to each other.

The embodiments are described with reference to the examples illustrated in the attached drawings. In the attached drawings, like reference numerals denote like or corresponding elements. In addition, in explaining the embodiments below, redundant description about like or corresponding elements may be omitted. However, even when description on an element is omitted, this is not intended to mean that the element is not included in a certain embodiment.

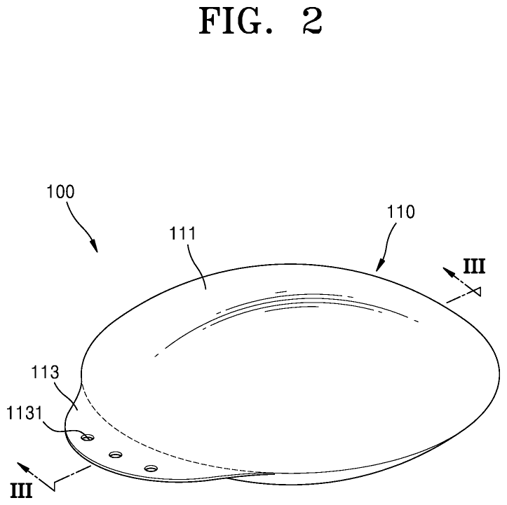

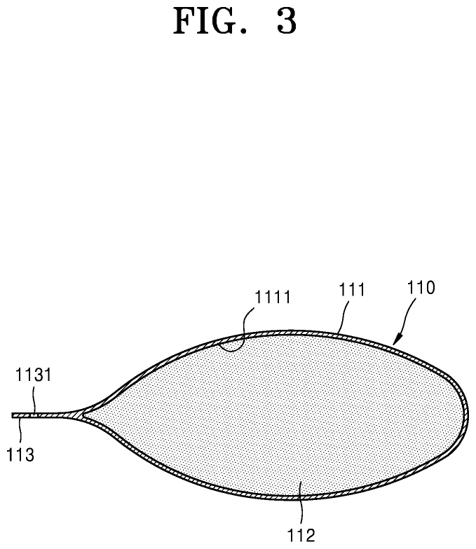

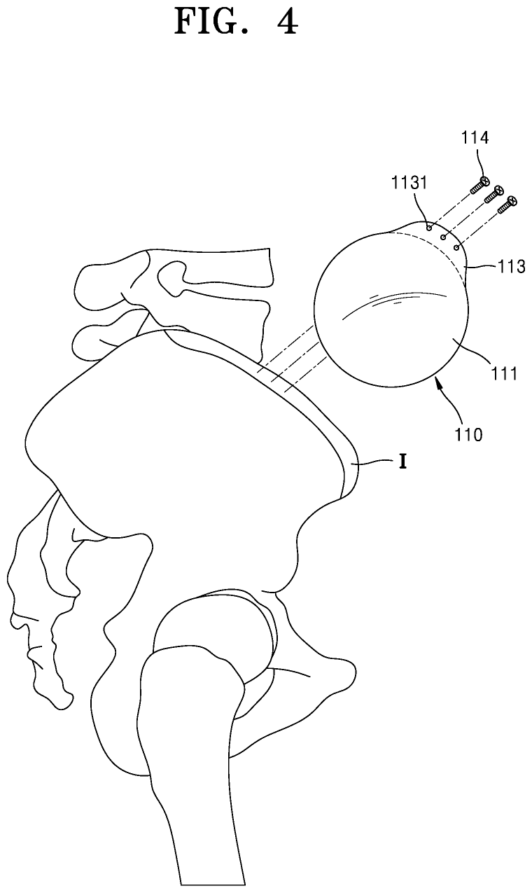

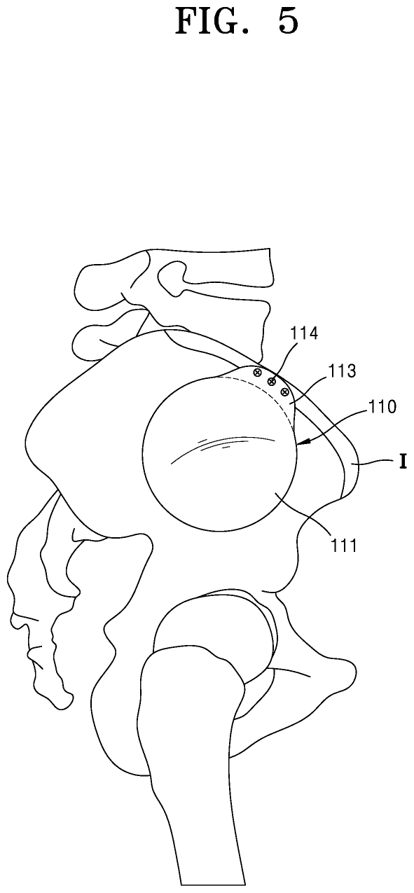

FIG. 2 is a perspective view illustrating a first embodiment of a first implant body of an implant for pelvic and hip plasty of the present disclosure, FIG. 3 is a cross-sectional view taken along line III-III in FIG. 2, FIG. 4 is a diagram illustrating placement of the first implant body of FIG. 2 onto a pelvis, and FIG. 5 is a diagram illustrating the first implant body of FIG. 2 placed onto a pelvis.

With reference to FIGS. 2 and 3, an implant for pelvic and hip plasty 100 according to a first embodiment may include a first implant body 110 fixed to an iliac crest of pelvis of a human and placed in hip muscles or between hip muscles and skin to increase the pelvis and hip volume.

The first implant body 110 may include an outer cover 111, a polymer gel 112, and a flange portion 113.

The outer cover 111 may have an interior space 1111, include an elastic material, and be placed in hip muscles or in body tissues such as muscles or skin tissues. A planar structure of the outer cover 111 may have a shape corresponding to a shape of hips into which the implant 100 is inserted. For example, the outer cover 111 may have an elliptical shape such as an egg shape and may increase the hip volume.

More specifically, the outer cover 111 may have a structure including an upper surface and a lower surface which are convex, and having a thickness which decreases gradually from the center towards the edge. However, the shape of the outer cover 111 is not limited to the foregoing. The upper surface of the outer cover 111 may have a convex shape while the lower surface has a flat shape, and the outer cover 111 may have various shapes.

The outer cover 111 may prevent exudation of contents in the interior space 1111, and silicon elastomer or polyurethane may be used as a materials of the outer cover 111. The outer cover 111 may selectively include other materials that are commonly used for an implant. In particular, the outer cover 111 may use a material having a low permeability. The outer cover 111 may include a single layer or multi-layers, and when the outer cover 111 includes multi-layers, the layers may include the same material, or different materials from each other.

The outer cover 111 may include various elastic areas, and the elastic areas may have the same thickness. However, some areas may have different thicknesses from each other.

The polymer gel 112 may be filled in the interior space 1111 of the outer cover 111 and may include a material that is harmless to human body. A silicon gel including silicon oil may be used as the polymer gel 112. However, a filler material filling the interior space 1111 of the outer cover 111 is not limited thereto, and polyurethane, or any other polymer gels may be used as the polymer gel 112.

The flange portion 113 may protrude from the edge of the outer cover 111 and may be in close contact with and fixed to an iliac crest I of a human. As the flange portion 113 does not include the polymer gel 112 therein, even when the flange portion 113 is damaged, no filling may leak to the outside. More specifically, the flange portion 113 may extends from a part of the edge of the outer cover 111.

The flange portion 113 may be integrated with the outer cover 111 and may include the same materials as the outer cover 111. That is, the flange portion 113 may be integrated with the outer cover 111 and fixed to the outer cover 111.

As the flange portion 113 include a material which has excellent elasticity and thus may be freely deformed in accordance with the shape of the iliac crest I, when the flange portion 113 is placed on the iliac crest I, the flange portion 113 may naturally have a shape corresponding to the shape of the iliac crest I. More specifically, the flange portion 113 may be fixed onto the iliac crest I, and may be deformable in accordance with the shape of the iliac crest I.

A first fastening hole 1131 may be formed in the flange portion 113. The fastening hole may be a hole through which a fixing screw 114 passes, and as a hole is formed, the fixing screw 114 may easily penetrate the flange portion 113 and be fixed to the iliac crest I. An operator may form a hole in the flange portion 113 on the spot when necessary; however, it may be better to form a hole in advance at a needed position in a factory.

The first fastening hole 1131 may be formed at the center of the flange portion 113. When the first fastening hole 1131 is formed close to the outer cover 111, peripheral region of the outer cover 111 may be damaged when the outer cover 111 is placed on the iliac crest I, which may lead to external leakage of the polymer gel 112 in the outer cover 111, and when the first fastening hole 1131 is formed close to the edge of the flange portion 113, the first fastening hole 1131 may be easily torn up.

There may be one first fastening hole 1131 or a plurality of first fastening holes 1131, and the first fastening hole 1131 may be added for reduced according to need.

The implant for pelvic and hip plasty 100 according to the first embodiment of the present disclosure may be placed as illustrated in FIGS. 4 and 5.

First, the skin around the hips of the human may be minimally incised. Then, while carefully examining the site under a microscope, a fixing groove into which the fixing screw 114 is inserted and fixed may be formed in the iliac crest I to which the implant for plasty 100 is to be fixed.

Next, the first implant body 110 may be inserted through the minimal incision, and the flange portion 113 may be in close contact with the iliac crest I. During this process, the outer cover 111 which contains the polymer gel 112 in the interior space 1111 may be placed between muscles or between the hip muscles and the skin.

Then, the first fastening hole 1131 of the flange portion 113 may be aligned with the pre-formed fastening groove, and the fixing screw 114 may be inserted and be finally fixed to the iliac crest I.

Although the present embodiments describes the case in which a fastening groove is formed in advance at the iliac crest I before the first implant body 110 is inserted into the human body, the technical ideas of the present disclosure is not limited thereto, and the fixing screw 114 may be drilled into the iliac crest I while the flange portion 113 of the first implant body 110 is in close contact with the iliac crest I.

When the fixing of the first implant body 110 to the iliac crest I is completed, the incision may be sutured to complete the surgery.

According to the implant for pelvic and hip plasty of the present disclosure, as the flange portion is separately provided at the edge on the outer cover and the fixing screw may be screwed in the flange portion, the implant for plasty may stay at the desired position in the hip tissues for a long time. That is, as the flange portion is fixed stably to the iliac crest, the outer cover containing the polymer gel may not sag over time, and may remain at the initial position where the implant is placed by the surgery. Thus, a re-operation is not required.

As the upper portion of the implant for plasty is fixed around the pelvis, and the lower portion of the implant is inserted into hip muscles, the volume of pelvis as well as the volume of hips may be increased.

In addition, according to the implant for pelvic and hip plasty of the present disclosure, as the flange portion does not contain the polymer gel therein, even when the flange portion is slightly damaged during the operation, external leakage of polymer gel may not happen.

Moreover, according to the implant for pelvic and hip plasty of the present disclosure, as the first fastening hole through which the fixing screw passes is formed in advance at the flange portion, an operator may easily penetrate the fixing screw and fix the fixing screw to the iliac crest, which leads to improved convenience in operation.

Furthermore, according to the implant for pelvic and hip plasty of the present disclosure, as the flange portion may be freely elastic-deformable in accordance with the shape of the iliac crest, the operator may properly bend and attach the flange portion onto the iliac crest, which also leads to increased convenience in operation.

The implant for pelvic and hip plasty according to the present disclosure may be modified as described below.

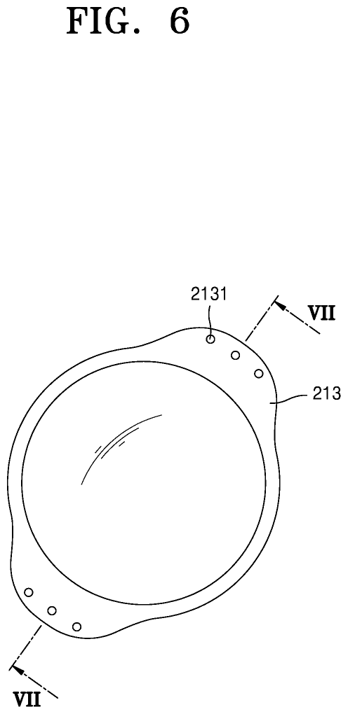

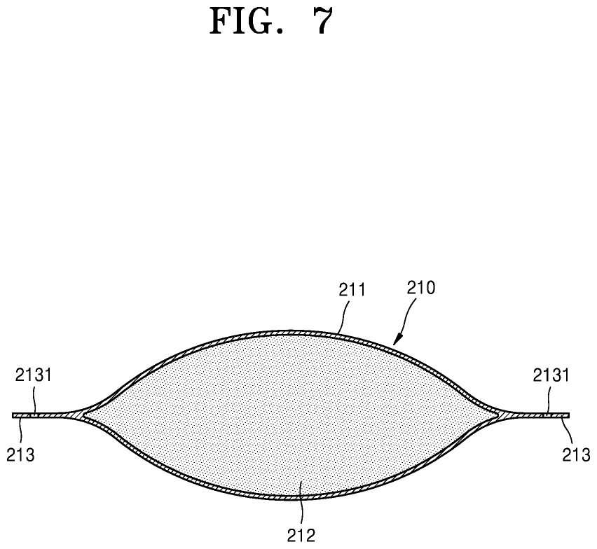

FIGS. 6 and 7 illustrate a first implant body 210 according to a second embodiment. Although the first embodiments illustrates that one flange portion 113 of the first implant body 110 protrudes from a part of the edge of the outer cover 111, the present disclosure is not limited thereto, and two flange portions 213 may protrude from an outer cover 211.

More specifically, two flange portions 213 may be formed at the edge of the outer cover 211, and the flange portions 213 may face each other with the outer cover 211 therebetween. When two flange portions 213 are formed at the outer cover 211, the outer cover 211 may be supported at multiple positions, which suggests that the flange portions 213 may provide more stable support.

However, the present disclosure is not limited thereto, and positions and numbers of the flange portion may vary according to need. That is, the positions and numbers of the flange portion may be properly changes according to shape and position of pelvis to which the implant is fixed.

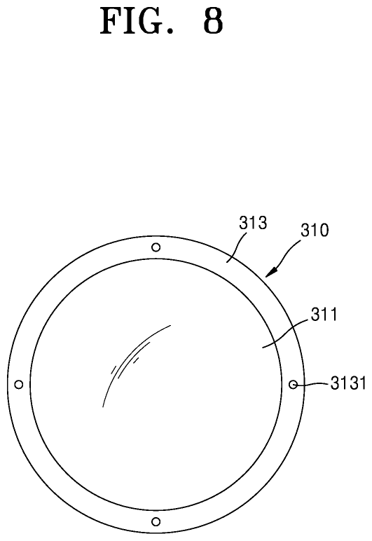

FIG. 8 illustrates a first implant body 310 according to a third embodiment. According to the first implant body 310 of a third embodiment, a flange portion 313 may extend from the entire edge of an outer cover 311 and form a ring shape. A plurality of first fastening holes 3131 may be formed at the flange portion 313, a fixing screw may be tightened properly in accordance with the position of pelvis to which the implant is fixed.

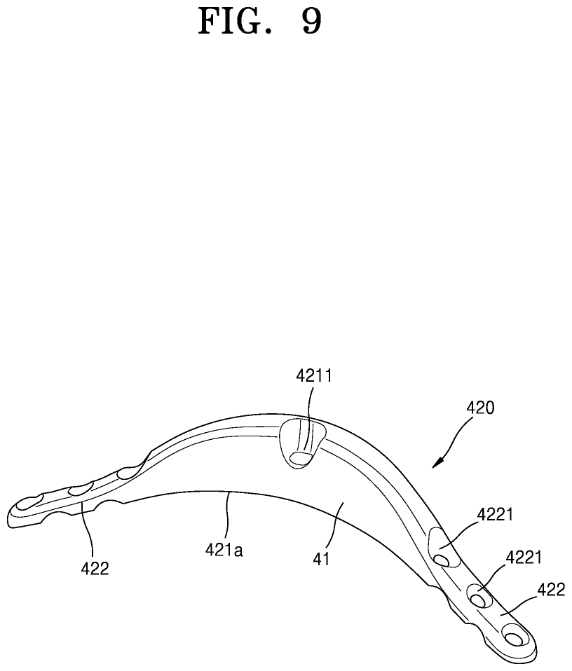

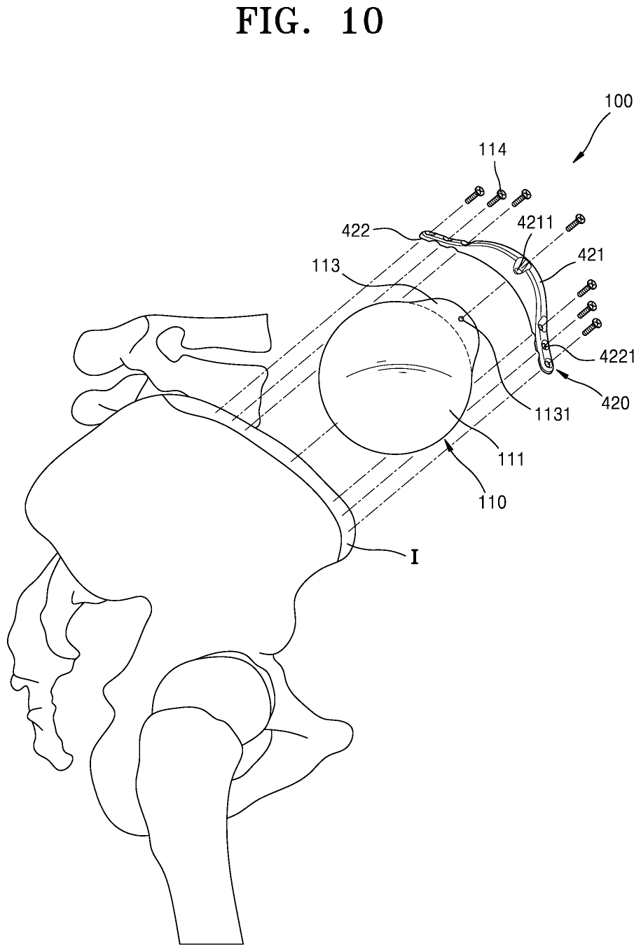

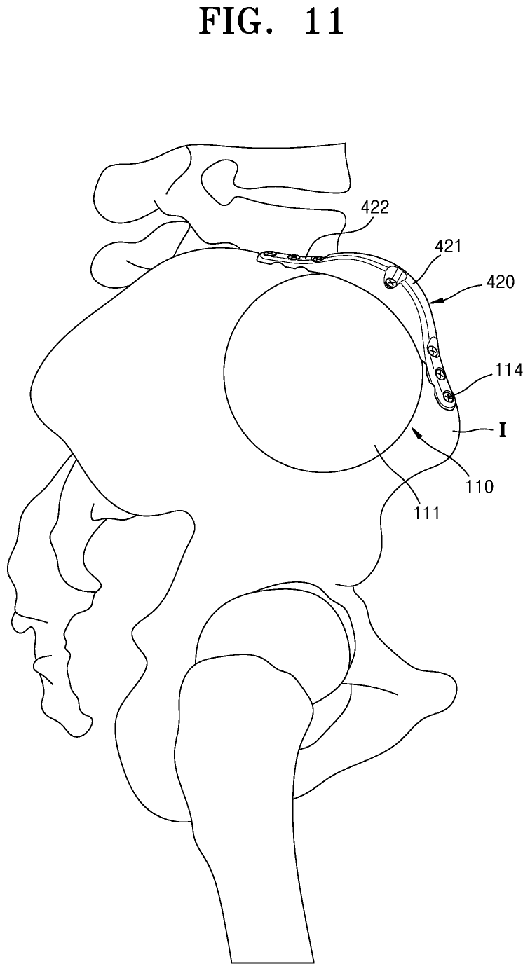

FIGS. 9 to 11 illustrate a second implant body 420. The second implant body 420 may be used along with the first implant body 110 and may be fixed to the iliac crest to increase the lateral width of the pelvis of the human. The second implant body 420 may include a base portion 421 and an extension 422.

The base portion 421 may be fixed to the iliac crest and increase the volume of the iliac crest area. A close contact surface 421a having a curved shape corresponding to the shape of the iliac crest may be formed at the bottom surface of the base portion 421. To shape the close contact surface 421a of the base portion 421 in accordance with the shape of the iliac crest, a three-dimensional (3D) modeling and 3D printing process may be performed on the pelvis of the human or average data regarding iliac crests of Korean women may be calculated. The second implant body 420 may need to be pre-manufactured based on the foregoing.

In the base portion 421, a second fastening hole 4211 may be formed at each position corresponding to the first fastening hole 1131 of the first implant body 110. The base portion 421 may be fixed to the flange portion 113 such that the flange portion 113 is pressured while the flange portion 113 of the first implant body 110 is in close contact with the iliac crest, and during this process, it may be necessary to place a second fastening hole 4211 at a position corresponding to the first fastening hole 1131 such that the fixing screw 114 penetrating the second fastening hole of the second implant body 420 passes through the first fastening hole 1131 formed at the flange portion 113 of the first implant body 110.

The extension 422 may extend from both sides of the base portion 421 and may have a thickness gradually deceasing from the base portion 421 towards an end portion of the extension 422.

The extension 422 may be provided to maintain the state in which the base portion 421 is securely fixed to the iliac crest and stably stays fixed without moving.

A third fastening hole 4221 through which the fixing screw 114 passes may be formed at the extension 422. The second implant body 420 may be securely fixed to the iliac crest by the fixing screw 114 placed to penetrate the third fastening hole 4221.

The base portion 421 and the extension 422 may include at least one material selected from a titanium alloy, a stainless alloy, polyethylene, and bio-ceramics. The base portion 421 and the extension 422 may also include a material such as polyether ether ketone, polymethyl methacrylate, etc.

A titanium alloy, a stainless alloy, polyethylene, and bio-ceramics are innoxuous and have elasticity and strength similar to those of human bones. Accordingly, these materials may have excellent in-vivo stability and biocompatibility. Thus, when the second implant body 420 including the foregoing materials is inserted in human body, side effect such as foreign body reaction, inflammation, etc. may be suppressed.

In addition, when a plurality of third fastening holes 4221 are provided, a plurality of fixing screws 114 may be provided as well, and the length of the plurality of fixing screws 114 may be identical. However, the technical ideas of the present disclosure is not limited thereto, and the length of the fixing screw 114 may vary.

A method of performing an operation using the first implant body 110 and the second implant body 420 is described below.

First, the skin around the hips of the human may be minimally incised. Then, while carefully examining the site under a microscope, a fixing groove into which the fixing screw 114 is inserted and fixed may be formed in the iliac crest to which the implant for plasty is to be fixed.

Next, the first implant body 110 may be inserted through the minimal incision, and the flange portion 113 may be in close contact with the iliac crest. During this process, the outer cover 111 which contains the polymer gel in the interior space may be placed between muscles or between the hip muscles and the skin.

The first fastening hole 1131 of the flange portion 113 may be aligned with a pre-formed fastening groove.

Then, the prepared second implant body 420 may be injected through the incision, and the second fastening hole 4211 may be aligned with the first fastening hole 1131. In addition, a third fastening screw may be aligned with the pre-formed fastening groove. Next, the fixing screw 114 may be inserted to finally fixe the second implant body 420 to the iliac crest. The incision may be sutured to complete the surgery.

As such, when the first implant body and the second implant body are fixed together to the iliac crest, the first implant body may be placed in hip muscles or between hip muscles and skin, and the second implant body may be fixed to the iliac crest to perform the function of increasing the lateral width of the pelvis.

That is, as the second implant body increases the lateral width of pelvis, the pelvis may become wider than waist, which emphasizes a slender waist.

In addition, the volume of hips may be increased by the first implant body, which leads to hip-up effect. In particular, as the second implant body is in close contact with the flange portion to support the flange portion, the second implant body may stay at its position more stably.

As the iliac crest is in a surface contact with the flange portion and receives pressure while the close contact surface of the second implant body is in a surface contact with the flange portion, the flange portion of the first implant body may be fixed to the iliac crest more conjunctly and securely, which prevents the first implant body from sagging or flowing down by gravity.

While embodiments has been particularly shown and described with reference to exemplary embodiments thereof and examples illustrated in the attached drawings, it will be understood by those of ordinary skill in the art that various substitutions, changes, and modifications may be made therein without departing from the spirit and scope of the present disclosure. Also, such substitutions, changes, and modifications should be considered as being included in the scope of claims.

1. An implant for pelvic and hip plasty for increasing pelvis and hip volumes of a human, the implant for pelvic and hip plasty comprising:
a first implant body including an outer cover which has an interior space, includes an elastic material, and is to be placed in hip tissues under an iliac crest of the human,
a polymer gel filled in the interior space of the outer cover, and
a flange portion projecting from an edge of the outer cover and including an elastic-deformable material to be in close contact with and fixed to the iliac crest of the human; and
a fixing screw penetrating the flange portion and inserted into the iliac crest to fix the flange portion to the iliac crest,
wherein, when the flange portion is in contact with the iliac crest, the flange portion deforms into a shape corresponding to an outer shape of the iliac crest and is in close contact with the iliac crest.

2. The implant for pelvic and hip plasty of claim 1,
wherein at least one first fastening hole is formed in the flange portion such that the fixing screw fixed to the iliac crest of the human passes through the at least one first fastening hole.

3. The implant for pelvic and hip plasty of claim 1,
wherein the flange portion is bendable to have a shape corresponding to a shape of the iliac crest.

4. The implant for pelvic and hip plasty of claim 1,
wherein the flange portion extends from an entire edge of the outer cover and forms a ring shape.

5. The implant for pelvic and hip plasty of claim 1,
wherein at least one flange portion extends from a part of the edge of the outer cover.

6. The implant for pelvic and hip plasty of claim 1,
wherein the flange portion includes a same material as the outer cover and is integrated with the outer cover.

7. The implant for pelvic and hip plasty of claim 1,
wherein the flange portion has a shape corresponding to the iliac crest and is fixed to the iliac crest.

8. The implant for pelvic and hip plasty of claim 1,
wherein the polymer gel includes a silicon gel.

9. The implant for pelvic and hip plasty of claim 1,
wherein the outer cover includes at least one of silicon elastomer and polyurethane.

10. The implant for pelvic and hip plasty of claim 1,
wherein the outer cover consists of an upper surface and a lower surface with respect to the flange portion, and the upper surface and the lower surface have a convex shape.

11. The implant for pelvic and hip plasty of claim 1, further comprising
a second implant body fixed to the iliac crest to extend a lateral width of a pelvis of the human and including a close contact surface having a shape corresponding to the iliac crest,
wherein the flange portion of the first implant body is inserted and fixed between the close contact surface of the second implant body and a surface of the iliac crest.

12. The implant for pelvic and hip plasty of claim 11,
wherein the second implant body includes:
a base portion fixed onto the iliac crest of the pelvis and increasing a volume of the iliac crest, and
an extension extending from an edge of the base portion.

13. The implant for pelvic and hip plasty of claim 12,
wherein a first fastening hole is formed in the flange portion, and
a second fastening hole is formed in the base portion of the second implant body at a position corresponding to the first fastening hole such that the fixing screw penetrates the first fastening hole and the second fastening hole and is fixed to the iliac crest to fix the first implant body and the second implant body to the iliac crest.

14. The implant for pelvic and hip plasty of claim 13,
wherein a third fastening hole through which the fixing screw passes is formed in the extension of the second implant body.

15. The implant for pelvic and hip plasty of claim 11,
wherein the second implant body includes at least one material selected from a titanium alloy, a stainless alloy, polyethylene, and bio-ceramics.

16. An implant for pelvic and hip plasty for increasing pelvis and hip volumes of a human, the implant for pelvic and hip plasty comprising:
a first implant body including an outer cover which has an interior space, includes an elastic material, and is to be placed in hip body tissues,
a polymer gel filled in the interior space of the outer cover, and
a flange portion which projects from an edge of the outer cover and is in close contact with and fixed to the iliac crest of the human; and
a second implant body fixed to the iliac crest to extend a lateral width of a pelvis of the human and including a close contact surface having a shape corresponding to the iliac crest,
wherein the flange portion of the first implant body is inserted and fixed between the close contact surface of the second implant body and a surface of the iliac crest.
